(19) United States
(12) Patent Application Publication
(10) Pub. No.: US 2004/0038419 A1
(43) Pub. Date: Feb. 26, 2004

(54) Assays and kits for detecting the presence of nitriles and/or cyanide

(75) Inventors: David Paul Weiner, Del Mar, CA (US); Jennifer Ann Chaplin, San Diego, CA (US); Ellen Chi, Del Mar, CA (US); Aileen Milan, San Diego, CA (US); Grace DeSantis, San Diego, CA (US); Mark J. Burk, San Diego, CA (US); Jeffrey McQuaid, San Diego, CA (US); Justin Stege, San Diego, CA (US)

Correspondence Address: FISH & RICHARDSON, PC, 12390 EL CAMINO REAL, SAN DIEGO, CA, 92130-2081, US

(73) Assignee: Diversa Corporation, San Diego, CA

(21) Appl. No.: 10/440,503

(22) Filed: May 15, 2003

Related U.S. Application Data
(60) Provisional application No. 60/380,737, filed on May 15, 2002.

(51) Int. Cl. G01N 33/00

(52) U.S. Cl. 436/109; 436/172

(57) ABSTRACT

In one aspect, invention relates to the field of monitoring chemical reactions and, in one aspect, provides methods and kits for detecting the presence of nitrites and/or cyanide. In one aspect, the invention provides methods and kits for assaying catalytic activity of a material, such as an enzyme, in a chemical or biochemical process, or assaying catalytic activity of a material in a biological sample, such as a whole cell.

### RELATED APPLICATIONS

This application claims the benefit of priority under 35 U.S.C. § 119(e) of U.S. Provisional Application No. 60/380,737, filed May 15, 2002. The aforementioned application is explicitly incorporated herein by reference in its entirety and for all purposes.

### TECHNICAL FIELD

This invention generally relates to organic and protein chemistry. In one aspect, invention relates to the field of monitoring chemical reactions and, in one aspect, provides methods and kits for detecting the presence of nitrites and/or cyanide. In one aspect, the invention provides methods and kits for assaying catalytic activity of a material, such as an enzyme, in a chemical or biochemical process, or assaying catalytic activity of a material in a biological sample, such as a whole cell.

### BACKGROUND

Assay techniques employing fluorogenic reagents to form a fluorescing complex are known to persons skilled in the art. One popular fluorogenic reagent for use in such reactions is o-phthalaldehyde (OPA). Under alkaline conditions, OPA forms 1-alkylthio-2-alkylisoindole (AAI), which is a highly fluorescent adduct. Naphthalene-2,3-dicarboxaldehyde (NDA) has also been used as a fluorogenic reagent.

Although fluorophores generated by the reaction of primary amines and OPA exhibit a relatively high intensity fluorescence, it has been observed that there are some significant drawbacks to the use of the OPA/thiol derivatizing system. For example, it is not useful for the detection of small quantities of peptides and proteins.

Another problem with fluorogenic assay techniques employing OPA relates to the relative instability of the 1,2-disubstituted isoindoles of certain amines such as glycine, γ-aminobutyric acid (GABA) and β-alanine. These adducts have been observed to readily degrade into non-fluorescent products, thereby placing severe time constraints on a practitioner carrying out an assay involving these materials, particularly when a concentration profile of one or more of the above-mentioned amine adducts is desired.

U.S. Pat. No. 4,758,520 (Matuszewski, et al.) discloses a chemiluminescence method for assaying compounds containing primary amino groups using 1-cyano-2-substituted benz(f)- or naphtha(f)-isoindole fluorescers. These fluorescers may be formed by reacting NDA with a primary amino group in the presence of cyanide ion.

U.S. Pat. No. 4,910,314 (de Montigny et al.) describes fluorescent adducts which are amenable to detection by fluorometric and electrochemical techniques. This patent also describes a method for assaying trace concentrations of analytes containing one or more primary amino groups or trace levels of cyanide wherein an aromatic dialdehyde is reacted with both cyanide ion and a primary amine in solution to yield an adduct which is detectable using fluorometric or electrochemical assaying techniques. NDA or OPA may be employed to form fluorescent adducts by reacting them with such primary amines and cyanide.

U.S. Pat. No. 5,631,374 (Novotny, et al.) discloses fluorescent derivatives of aroyl-2-quinoline-carboxaldehyde and their use in the detection and quantification of minute amounts of primary amines. In one embodiment, the fluoroscers are formed in the presence of cyanide ions to assess the effect of the concentration of cyanide ions on reaction yield.

Jallegeas et al. have also developed an assay for cyanide, α-aminonitriles, and α-hydroxynitriles for the study of the biological hydrolysis of these compounds. See Jallegeas et al., Development of an Assay Method for Cyanide, α-Aminonitriles, and α-Hydroxy nitrites for the Study of the Biological Hydrolysis of These Compounds, ANALYST (London), 109(11), 1439-42(1984). However, this assay method is not suitable to be implemented in a high throughput setting.

Industry has recognized the need for new enzymes for a wide variety of applications. As a result, a variety of microorganisms have been screened to ascertain whether or not such microorganisms have a desired activity. If such microorganisms have the desired activity, the enzyme in the microorganism, which is responsible for the activity, may then be recovered from the microorganism.

Many assays have been developed to screen enzymes based on their biological activities. However, most assays involve complicated processes and detection equipment. The complexity of these assays make them difficult to employ in high throughput screening thereby limiting the number of candidates that can be screened in a given time period.

In addition, for certain industrial processes, more sophisticated methods for monitoring reaction progress are desired. This is particularly true for chemical or enzymatic synthesis processes where small amounts of the desired product are produced. This is also true for processes, which produce specialized chemical products.

### SUMMARY

In one aspect, the invention provides methods and kits for monitoring a chemical or biochemical process, which employs, as a reactant, cyanide or a material that can be readily converted to cyanide, or which generates, as a reaction product, cyanide or a material that can be readily converted to cyanide. In the method, the reaction is monitored by sampling the reactants or products, converting to cyanide, if necessary, and measuring the cyanide concentration using fluorescence detection techniques.

In one aspect, the invention relates to methods and kits for assaying catalytic activity in a chemical or biochemical process, which employs, as a reactant, cyanide or a material that can be converted to cyanide, or which generates, as a reaction product, cyanide or a material that can be converted to cyanide. In the method, the reaction is monitored by sampling the reactants or products, converting the reactant or product in the sample to a cyanide, if necessary, and measuring the cyanide concentration using any fluorescent detection technique. From the cyanide concentration, the degree of conversion of the reaction can be determined and from this the catalytic activity of a material present during the reaction can be assessed.

In one aspect, the invention provides methods and kits for screening biological samples for a particular activity. The method includes the steps of: combining the biological sample with a suitable substrate to form a reaction mixture, conducting a reaction in the reaction mixture, contacting the resultant reaction mixture with a derivatizing agent and an amine for a suitable period of time to form a fluorescent compound; and detecting the fluorescence of the fluorescent compound to determine if the biological sample has the particular activity of interest.

In an alternative aspect of any of any method of the invention, the reaction is quenched to produce a cyanide-containing reaction mixture and the cyanide is employed to form the fluorescent compound in order to detect the concentration of the cyanide in the reaction mixture.

In one aspect, the invention relates to a kit for determining if a biological sample has a particular activity. The kit includes a suitable substrate to be combined with the biological sample to form a reaction mixture, a derivatizing agent and an amine to be contacted with the reaction mixture to generate a fluorescent compound.

The invention provides methods for monitoring a chemical or biochemical process, comprising the following steps: (a) providing a reactant comprising a cyanide or a material that can be converted to a cyanide, or, a reactant that generates as a reaction product a cyanide or a material that can be converted to cyanide; (b) reacting the reactant and monitoring the reaction by sampling the reactant or a product and, if the reactant is a material that can be converted to a cyanide or the reactant generates a material that can be converted to cyanide, converting the reactant or the product to a cyanide; and (c) measuring the cyanide concentration in the sample, thereby monitoring the chemical or biochemical process.

The invention provides methods for assaying catalytic activity of a material in a chemical or biochemical process, or a material in a biological sample, comprising the following steps: (a) providing a reactant comprising a cyanide or a material that can be converted to a cyanide, or, a reactant that generates as a reaction product a cyanide or a material that can be converted to cyanide; (b) reacting the reactant and monitoring the reaction by sampling the reactant or a product and, if the reactant is a material that can be converted to a cyanide or the reactant generates a material that can be converted to cyanide, converting the reactant or the product to a cyanide; and (c) measuring cyanide concentration in the sample and determining the degree of conversion of the reaction from the cyanide concentration, thereby assaying the catalytic activity of the material during the reaction.

In one aspect of the methods of the invention, the cyanide concentration in the sample is measured by using a chemical technique. The cyanide concentration can be measured by derivatization of the cyanide with a fluorescing agent. The cyanide concentration in the sample can be measured by using a fluorescence detection technique. In alternative aspects, the fluorescing agent comprises a naphthalene-2,3-dialdehyde (NDA) or equivalent, anthracene dicarboxyaldehyde (ADA) or equivalent, or, the fluorescing agent comprises an o-phthalaldehyde (OPA) or equivalent. The o-phthalaldehyde (OPA) can be substituted at one or both of the 4 and 5 positions with substituents capable of enhancing the stability and fluorescence quantum of an isoindole product. The substituents can comprise a methoxy substituent, a dimethylamino substituent or both.

In one aspect, the fluorescing agent comprises a composition having a formula comprising

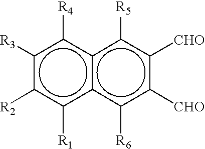

or variations thereof wherein one or more aromatic carbons are substituted with a heteroatom or a hetero group and/or one or both of the CHO groups can be a —COR group, wherein R is selected from the group consisting of an alkyl group, an aryl group or an alkoxy group, wherein the substituents R₁, R₂, R₃, R₄, R₅, R₆ are selected from one of the following:

(A) R₁ is selected from —H, an alkyl group, an aryl group, —N(CH₃)₂, —SO₃H, —NO₂, —SO₃⁻Na⁺ and

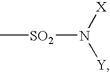

wherein X and Y may be the same or different and are independently selected from —H, an alkyl group, an aryl group and C₁-C₈ alkyl groups, and R₂-R₆ are —H;

(B) R₁, R₄, R₅ and R₆ are —H, an alkyl group or an aryl group, and R₂ and R₃ are combined to form one of:

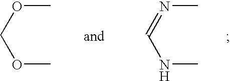

(C) R₁ and R₄ are independently selected from an alkyl group, an aryl group, —N(CH₃)₂ and

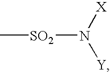

and R₂, R₃, R₅ and R₆ are —H, an alkyl group or an aryl group;

(D) R₁, R₂, R₃, and R₄ are —H, an alkyl group, an aryl group, and R₅ and R₆ are independently selected from an alkyl group, an aryl group, —OCH₃,

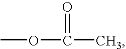

—OSi(CH₃)₂C₄H₉, and N(CH₃)₂; or

(E) R₁, R₄, R₅, and R₆ are —H, and R₂ and R₃ are independently selected from an alkyl group, an aryl group, —CH₃O,

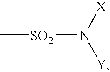

—SO₃H, —CO₂H, and salts thereof; and

(F) R₁, R₃, R₄, R₅ and R₆ are H an alkyl group or an aryl group, and R₂ is —(CH₃)₂N.

In one aspect, the heteroatom or hetero group comprises a nitrogen, an oxygen, a sulfur, a mercapto group, a thia group, a thio group, an aza group or an oxo group.

In one aspect, the fluorescing agent is reacted with a cyanide-containing mixture using a stoichiometric excess of amine and used to measure cyanide concentration. In one aspect, the fluorescing agent is reacted with a degradation product of a substrate to form a compound that can be detected using a spectrometer, e.g., a fluorometer, an IR spectrometer or a UV spectrometer. In one aspect, the fluorescing agent is reacted with a cyanide starting material or a reaction product, or a product of the reaction can be converted to cyanide for reaction with the fluorescing agent, to determine conversion due to a hydrolysis reaction.

In one aspect, the fluorescing agent comprises a compound selected from the group consisting of:

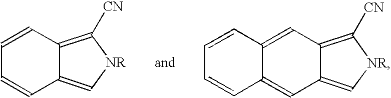

wherein R is an H—, an alkyl or an aryl group.

In one aspect, the fluorescing agent comprises a 3-(4-carboxybenzoyl)quinoline-2-carboxaldehyde (CBQCA, ATTO-TAG™) (Molecular Probes, Inc., Eugene, Oreg.) or equivalent. The optimal wavelength for excitation of products produced from 3-(4-carboxybenzoyl)quinoline-2-carboxaldehyde (CBQCA) or equivalent is about 488 nm, and the optimal wavelength for emission is about 570 nm. In one aspect, the 3-(4-carboxybenzoyl)quinoline-2-carboxaldehyde (CBQCA) or equivalent is used in solution at a concentration of between about 10⁻¹⁵ mole/liter (M) to about 1 mole/liter (M). In one aspect, the reaction is quenched to produce a cyanide-containing reaction mixture.

In one aspect, the reactant comprises a substrate for a reaction. The substrate can comprise a cyanide, a nitrile-group containing compound or a mixture thereof. The substrate can comprise an a-hydroxynitrile, an aminonitrile and/or mixtures thereof. The substrate can comprise a hydroxymethyl thiobutyronitrile (HMTBN), a lactonitrile, a propionaldehyde cyanohydrin (PAC), a 2-chloromandelonitrile (CMN), a cyclohexylmandelonitrile (CHMN), an acetophenone aminonitrile (APA), a phenylglycine (PGN), a dimethylbutanal aminonitrile (DMB), a hydroxylpivaldehyde aminonitrile (HPA), a pivaldehyde aminonitrile (PAH), mandelonitrile (MN) and/or mixtures of two or more of these compounds.

In one aspect, the substrate undergoes reactions with high rate constants or reactions which favor relatively high conversion to cyanide when the conversion of a substrate to cyanide involves an equilibrium reaction. In one aspect, the reaction is under alkaline conditions to quench chemical hydrolysis of the substrate. In one aspect, the substrate is used as a solution with a concentration of between about 10⁻¹⁵ mole/liter (M) to about 10 mole/liter (M), between about 1 μM to about 1 mole/liter (M), between about 1 μM to about 100 μM, between about 100 μM to about 100 μM, or between about 10 mM to about 500 mM.

In one aspect, the substrate is in an aqueous solution at a substrate concentration of between about 30 mM to about 150 mM. In one aspect, the fluorescing agent is used in the form of a solution. In one aspect, the fluorescing agent is used in the form of a solution with a concentration of between about 10⁻¹⁵ mole/liter (M) to about 1 mole/liter (M), between about 1 μM to about 500 mM, between about 1 μM to about 100 mM, or between about 30 mM to about 100 mM.

In one aspect, the fluorescing agent further comprises a buffer to control pH. In one aspect, the pH range of the solution of fluorescing agent is between about pH 8.5 and about pH 12.5, or between about pH 10 and about pH 11. In one aspect, in order to determine cyanide concentration a sample containing cyanide is added to a fresh, buffered pH-controlled solution of about pH 7 to about 10. The buffered pH-controlled solution can comprise an aromatic dicarboxaldehyde, e.g., at about 1 to about 500 millimolar aromatic dicarboxaldehyde, or, the buffered pH-controlled solution can comprise a primary amine, e.g., about 1 to about 1000 millimolar primary amine. The primary amine can be in a buffered pH-controlled solution, e.g., of about pH 7 to about 10 at a temperature, e.g., ranging from about 25° C. to about 40° C.

In one aspect, the reaction is allowed to continue for about 10 seconds to about 1 week, or, for about 10 minutes to about one hour. After the reaction has gone substantially to completion the concentration of cyanide can be determined by measuring amounts of adducts in the solution using high performance liquid chromatography (HPLC) with a fluorescence or a chemi-luminescence detection technique.

In one aspect, the optimal wavelengths for excitation of the products produced from o-phthalaldehyde (OPA) or equivalent are 230 nm and about 320-340 nm, and the optimal wavelength for emission is about 375-385 nm. The optimal wavelengths for excitation of the products produced from naphthalene-2,3-dialdehyde (NDA) or equivalent are about 250 nm, 420 nm or 450 nm and the optimal emission wavelength is about 490 nm.

The invention provides methods for screening a biological sample for a particular activity, comprising the following steps: (a) providing a substrate, a derivatizing agent and an amine; (b) providing a biological sample; (c) combining the biological sample with the substrate to form a reaction mixture, thereby conducting a reaction in the reaction mixture; (d) contacting the resultant reaction mixture with the derivatizing agent and the amine for a suitable period of time to form a fluorescent compound; and (e) detecting the fluorescence of the fluorescent compound to determine the activity of interest in the biological sample. In one aspect, the amines comprises a primary amine or an amino acid. In one aspect, the amine comprises an alkylamine, an arylamines. In one aspect, the amine or amino acid comprises a glycine, an alanine, a tyrosine, a valine, a phenylalanine, an aspartic acid, a glutamic acid, a cysteic acid, a serine, a histidine, a threonine, an isoleucine, a methionine, a tryptophan, an arginine, an asparagine, a GABA, an n-acetyl lysine or a glutamine.

In one aspect, the method is used as a screening technique to screen for a particular enzymatic or catalytic activity. In one aspect, the activity of interest is an activity of a catalyst which catalyzes the hydrolysis of nitrile groups in nitrile-group containing compounds. In one aspect, the catalyst is employed in a hydrolysis reaction and the reaction is quenched and the amount of nitrile-group containing compound remaining in the reaction mixture is determined. In one aspect, the activity comprises enzymatic hydrolysis of nitrile-group containing compounds.

In one aspect, the methods comprise the steps of: contacting a biological sample with a suitable nitrile group-containing substrate in the presence of water to cause hydrolysis of at least some of the nitrile-groups in the substrate, quenching the reaction to a pH of about 10 to about 12, or, about pH 10 to about 11, to stop the hydrolysis reaction and decompose at least a portion of the remaining nitrile-group containing compound to produce cyanide, contacting the cyanide-containing mixture with a fluorescing agent for a suitable period of time to form a fluorescent compound; detecting the concentration of the fluorescent compound, and calculating the concentration of the nitrile group-containing substrate remaining in the reaction mixture to determine if the biological sample has the desired activity. In one aspect, the final step of the method further comprises measuring the fluorescence intensity emitted from a fluorescent compound; comparing the measured fluorescence, the concentration of cyanide in the sample, and determining the activity of a biological sample based on the amount of cyanide in the sample by relating the amount of cyanide to the degree of conversion of the nitrile-group containing starting material. In one aspect, negative control samples and/or positive control samples are assayed with test samples to provide baselines for determining which biological samples have a desired activity.

In one aspect, the method comprises assaying the catalytic activity of a material in a biological sample. The biological sample can be derived from an environmental sample, a sample containing more than one organism, a sample comprising a mixed populations of organisms, an enriched sample, a sample from an isolated organism, a sample comprising a cultured organism or a sample comprising an uncultured organism. The biological sample can comprise a microorganism existing in nature, a microorganism isolated from nature, a microorganism from a library, a clone from a library, an enzyme, a materials containing an enzyme, a cell, a DNA molecule, an RNA molecule or a living organism. In one aspect, the biological sample comprises a microorganism, a whole cell, an enzymes and/or a clone that comprises a sample from a mixed population library. The biological sample can comprise a whole cell suspension or a clone from a mixed population library. In one aspect, the mixed population library is derived from a mixed population of organisms. The mixed population of organisms can be derived from an environmental sample or an uncultivated population of organisms or a cultivated population of organisms.

In one aspect, the method comprises use of a high throughput screening method, such as a microarray or a fluorescence activated cell sorting device (FACS). The microarray can be GIGAMATRIX™ (Diversa Corporation, San Diego, Calif.).

In one aspect, the catalyst which catalyzes the hydrolysis of nitrile groups in nitrile-group containing compounds is an enzymatic activity that catalyzes the hydrolysis of a compound selected from α-hydroxynitriles and aminonitriles. The catalyst which catalyzes the hydrolysis of nitrile groups in nitrile-group containing compounds can be a nitrilase. In one aspect, the nitrilase comprises a nitrile hydratase, a hydroxynitrile lyase, or an oxynitrilase. The nitrilase can comprise a sequence as set forth in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384 or 386. The nitrilase can comprise a polypeptide encoded by a nucleic acid sequence as set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 374, 381, 383 or 385.

In one aspect, the method is performed in a whole cell environment, or, with cell lysates or cell extracts, or, a combination thereof.

In one aspect, the fluorescence detection technique comprises a fluorescence polarization, a time-resolved fluorescence, FRET, fluorescence activated cell sorting (FACS), HPLC or capillary electrophoresis (CE) technique.

The invention provides kits for determining if a biological sample has a particular activity comprising a substrate to be combined with the biological sample to form a reaction mixture, a derivatizing agent and an amine to be contacted with the reaction mixture to generate a fluorescent compound. The derivatizing agent in the kits can comprise a fluorescing agent, e.g., a naphthalene-2,3-dialdehyde (NDA) or equivalent, or an o-phthalaldehyde (OPA) or equivalent. The o-phthalaldehyde (OPA) can be substituted at one or both of the 4 and 5 positions with substituents capable of enhancing the stability and fluorescence quantum of an isoindole product. The substituents can comprise a methoxy substituent, a dimethylamino substituent or both.

In one aspect, the fluorescing agent comprises a 3-(4-carboxybenzoyl)quinoline-2-carboxaldehyde (CBQCA, ATTO-TAG™), or, a composition having a formula comprising

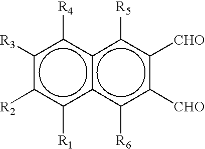

or variations thereof wherein one or more aromatic carbons are substituted with a heteroatom or a hetero group and/or one or both of the —CHO groups can be a —COR group, wherein R is selected from the group consisting of an alkyl group, an aryl group or an alkoxy group,

wherein the substituents R₁, R₂, R₃, R₄, R₅, R₆ are selected from one of the following:

(A) R₁ is selected from —H, an alkyl group, an aryl group, —N(CH₃)₂, —SO₃H, —NO₂, —SO₃⁻Na⁺ and

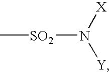

wherein X and Y may be the same or different and are independently selected from —H, an alkyl group, an aryl group and C₁-C₈ alkyl groups, and R₂-R₆ are —H;

(B) R₁, R₄, R₅ and R₆ are —H, an alkyl group or an aryl group, and R₂ and R₃ are combined to form one of:

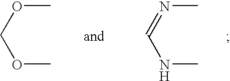

(C) R₁ and R₄ are independently selected from an alkyl group, an aryl group, —N(CH₃)₂ and

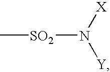

and R₂, R₃, R₅ and R₆ are —H, an alkyl group or an aryl group;

(D) R₁, R₂, R₃, and R₄ are —H, an alkyl group, an aryl group, and R₅ and R₆ are independently selected from an alkyl group, an aryl group, —OCH₃,

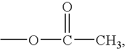

—OSi(CH₃)₂C₄H₉, and N(CH₃)₂; or

(E) R₁, R₄, R₅, and R₆ are —H, and R₂ and R₃ are independently selected from an alkyl group, an aryl group, —CH₃O,

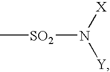

—SO₃H, —CO₂H, and salts thereof; and

(F) R₁, R₃, R₄, R₅ and R₆ are H an alkyl group or an aryl group, and R₂ is —(CH₃)₂N.

In one aspect, the heteroatom or hetero group comprises a nitrogen, an oxygen, a sulfur, a mercapto group, a thia group, a thio group, an aza group or an oxo group.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates a calibration curve for an assay of the present invention using hydroxymethyl thiobutyronitrile (HMTBN) as a substrate.

FIG. 2 illustrates a calibration curve for an assay of the present invention using lactonitrile as a substrate.

FIG. 3 illustrates a calibration curve for an assay of the present invention using propionaldehyde cyanohydrin as a substrate.

FIG. 4 illustrates calibration curves for assays of 2-chloromandelonitrile (CMN), cyclohexylmandelonitrile (CHMN), acetophenone aminonitrile (APA), and phenylacetaldehyde cyanohydrin (PAC) substrates.

FIG. 5A illustrates detection curves for assays of pivaldehyde aminonitrile (PAH), PAH combined with a lyophilized nitrilase lysate having a SEQ ID NO: 190 (“PAH+SEQ ID NO: 190”), acetophenone aminonitrile (APA), and APA combined with a lyophilized nitrilase lysate having a SEQ ID NO: 190 (“APA+SEQ ID NO: 190”), all using OPA as the fluorogenic reagent.

FIG. 5B illustrates detection curves for PAH, PAH combined with a lyophilized nitrilase lysate having a SEQ ID NO: 190 (“PAH+SEQ ID NO: 190”), APA, and APA combined with a lyophilized nitrilase lysate having a SEQ ID NO: 190 (“APA+SEQ ID NO: 190”), all using naphthalene dicarboxyaldehyde (NDA) as the fluorogenic reagent.

FIG. 5C illustrates detection curves for hydroxylpivaldehyde aminonitrile (HPA), HPA combined with a nitrilase of SEQ ID NO: 56(“HPA+SEQ ID NO: 56”), dimethylbutanal aminonitrile (DMB), and DMB combined with a nitrilase of SEQ ID NO: 60 (“DMB+SEQ ID NO: 60”), all using OPA as the fluorogenic reagent.

FIG. 5D illustrates detection curves for HPA, HPA combined with a nitrilase of SEQ ID NO: 56 (“HPA+SEQ ID NO: 56”), DMB, and DMB combined with a nitrilase of SEQ ID NO: 60 (“DMB+SEQ ID NO: 60”), all using NDA as the fluorogenic reagent.

FIG. 6A illustrates detection curves for DMB, DMB combined with a lyophilized nitrilase lysate of SEQ ID NO: 60 (“DMB+SEQ ID NO: 60”), APA, and APA combined with a lyophilized nitrilase lysate having a SEQ ID NO: 190 (“APA+SEQ ID NO: 190”), all using OPA in a sodium phosphate buffer as the fluorogenic reagent.

FIG. 6B illustrates detection curves for PAH, PAH combined a lyophilized nitrilase lysate having a SEQ ID NO: 190 (“PAH+SEQ ID NO: 190”), HPA, and HPA combined with a lyophilized nitrilase lysate of SEQ ID NO: 56 (“HPA+SEQ ID NO: 56”), all using OPA in a sodium phosphate buffer as the fluorogenic reagent.

FIG. 7 illustrates detection curves for the hydrolysis of mandelonitrile with unlysed whole cells (expressing a nitrilase having SEQ ID NO: 188), with whole cells having been lysed in in-situ with B-PER from Pierce (a nitrilase having SEQ ID NO: 188+BP), and with lyophilized cell lysate containing a nitrilase having SEQ ID NO: 188.

FIG. 8A illustrates detection graphs for hydrolysis of whole-cell mandelonitrile in a single-plate format using an assay of the present invention.

FIG. 8B illustrates detection curves for the whole cell hydrolysis of mandelonitrile, further data for which is shown in FIG. 9A, at the six-hour time point using an assay of the present invention.

FIG. 9A is one comparison of the conversion of CMN as a result of hydrolysis as determined by HPLC versus the conversion determined by an OPA assay of the present invention at the two-hour time point.

FIG. 9B is a second comparison of the conversion of CMN as a result of hydrolysis as determined by HPLC versus the conversion determined by an OPA assay of the present invention at the two-hour time point.

FIG. 9C is a comparison of the conversion of PAC as a result of hydrolysis as determined by HPLC versus the conversion determined by an OPA assay of the present invention at the two-hour time point.

FIG. 10 illustrates the results of a primary screening of Example 3 on clones of an expression library according to the present invention.

FIG. 11 illustrates the results of a secondary confirmation screening of Example 3 according to the present invention.

FIG. 12 illustrates an exemplary enzymatic hydrolysis reaction using a nitrilase enzyme.

FIG. 13 illustrates an exemplary enzymatic hydrolysis reaction using a nitrilase enzyme.

FIG. 14 illustrates an exemplary derivatization reaction for measuring cyanide concentration by derivatization of the cyanide with the fluorescing agent o-phthalaldehyde (OPA) to give a fluorescent product.

FIG. 15 illustrates an exemplary derivatization reaction for measuring cyanide concentration by derivatization of the cyanide with the fluorescing agent naphthalene-2,3-dialdehyde (NDA) to give a fluorescent product.

FIG. 16 illustrates CBQCA reacting with a primary amine and a cyanide group, as discussed in detail, below.

FIG. 17 illustrates the structures of exemplary fluorescing agents that can be used in the kits and methods of the invention.

Like reference symbols in the various drawings indicate like elements.

### DETAILED DESCRIPTION

In one aspect, the invention relates to methods and kits for monitoring the progress of a reaction. In one aspect, the invention takes advantage of the fact that certain reactions or compounds produce cyanide under controlled reaction conditions. The cyanide can be employed to form a fluorescent compound in order to detect the concentration of the cyanide in a sample or a reaction mixture. The methods of the present invention can be employed to monitor the reaction progress of any reaction.

In one aspect, the methods and kits of the invention employ as a reactant, or produce, as a reaction product, a cyanide or a compound, which reacts to produce cyanide under controlled reaction conditions. The methods and kits of the present invention can employ fluorometric and/or chemical techniques to detect the presence of, and an amount of cyanide, in order to monitor the progress of certain reactions.

In one aspect, the invention provides methods and kits for monitoring industrial processes involving certain chemical or enzymatic reactions. The invention also provides a simple method for determining the degree of conversion in chemical or enzymatic processes involving certain chemical starting materials or reaction products. In one aspect, the invention provides an assay for screening to identify enzymatic activity in the enzymatic conversion of certain chemical starting materials to other compounds. In one aspect, the invention provides methods for screening to identify catalytic activity in the chemical conversion of certain chemical starting materials to other compounds.

Naturally occurring assemblages of microorganisms often encompass a bewildering array of physiological and metabolic diversity. Therefore, in one aspect, the invention provides a method to screen for a desired enzymatic activity from an array of microorganisms to determine if a particular microorganism contains an enzyme with the desired activity in the conversion of certain chemical starting materials to other compounds.

In one aspect, the invention provides kits that, inter alia, can be used to screen biological samples to identify samples with a desired activity in the conversion of certain chemical starting materials to other compounds. In one aspect, a reactant for the reaction of interest is the substrate for a reaction. The substrate can be a cyanide or a compound that can be reacted or decomposed to produce cyanide. Exemplary substrates include, but are not limited to, cyanides, and nitrile-group containing compounds. A substrate can comprise an a-hydroxynitrile, an aminonitrile and/or mixtures thereof. Exemplary substrate compounds can comprise hydroxymethyl thiobutyronitrile (HMTBN), lactonitrile, propionaldehyde cyanohydrin (PAC), 2-chloromandelonitrile (CMN), cyclohexylmandelonitrile (CHMN), acetophenone aminonitrile (APA), phenylglycine (PGN), dimethylbutanal aminonitrile (DMB), hydroxylpivaldehyde aminonitrile (HPA), pivaldehyde aminonitrile (PAH), mandelonitrile (MN), and/or mixtures of two or more of these compounds.

Any compound that produces a predictable quantity of cyanide under controlled reaction conditions can be used as a substrate in a process of the present invention. Alternatively, the compound that produces a predictable quantity of cyanide can be a product of the reaction to be monitored if cyanide is a reaction product of the reaction.

The substrate is chosen for the ease with which it can be reacted to produce cyanide and on the basis of the amount of cyanide it will produce in a given time period. Thus, for example, when the conversion of a substrate to cyanide involves an equilibrium reaction, for example, substrates which undergo reactions with high rate constants or reactions which favor relatively high conversion to cyanide can be used since this will reduce the sampling and detection time and/or permit lower quantities of reactants to be detected within the detection limits of the assay. Thus, in this aspect, compounds including HMTBN, lactonitrile, propionaldehyde cyanohydrin, PGN, CMN, CHMN, APA and mandelonitrile are favored substrates since these substrates, or reaction products derived from these substrates, can be quickly and easily decomposed to produce detectable amounts of cyanide. In one aspect, the reaction is under alkaline conditions that can also serve to quench the chemical hydrolysis of these substrates. This permits use of the assay of the present invention in a very high throughput screening process since numerous measurements can be made in a short time period due to the ease of quenching the reaction and producing detectable amounts of cyanide in the reaction mixture.

In one aspect, the substrate is used as a solution with a concentration of about 10 millimole/liter (mM) to about 10 mole/liter (M), or, the substrate is used as a solution with a concentration of about 10 millimole/liter (mM) to about 500 mM, or, the substrate is in an aqueous solution at a substrate concentration of about 30 mM to about 150 mM.

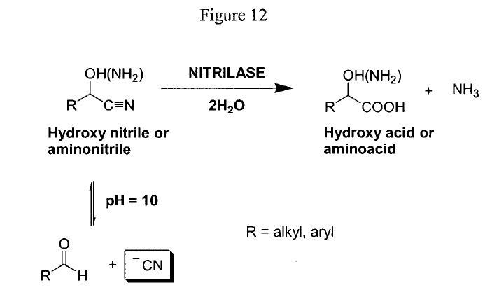

An exemplary enzymatic hydrolysis reaction using a nitrilase enzyme which can be used as the basis for the assay in accordance with the present invention is depicted in FIG. 12.

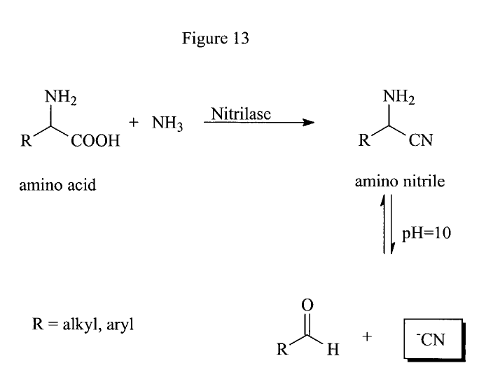

In another aspect, the reverse reaction of the enzymatic reaction of FIG. 12 may also be used as the basis for the assay in accordance with the present invention as in FIG. 13. Reaction progress can be monitored by monitoring the concentration of the α-hydroxynitrile or aminonitrile reactant. Thus, after a given reaction period, the enzymatic hydrolysis reaction can be quenched by raising the pH to at least 10, in this case. At pH of about 10-12, the remaining substrate will decompose via an equilibrium reaction to produce cyanide. If the equilibrium constant of the decomposition reaction is known, or a calibration curve has been prepared, the concentration of substrate remaining can be calculated from the measured concentration of cyanide in the quenched reaction mixture.

Alternatively, when the assay or kit of the present invention is employed for screening materials that catalyze the reaction of interest, certain threshold cyanide concentrations can be pre-selected. Then, for example, all measurements wherein the cyanide concentration is below the pre-selected threshold can be identified as positive hits since these samples demonstrate the greatest degree of conversion of the substrate to the hydrolysis products. From this, it can be concluded that a material is a good candidate for the catalysis of the reaction of interest.

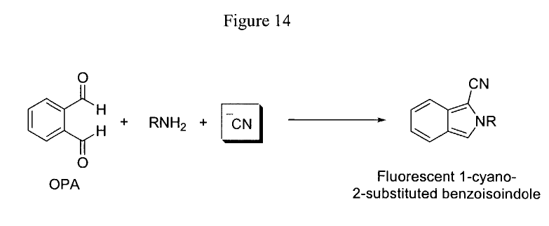

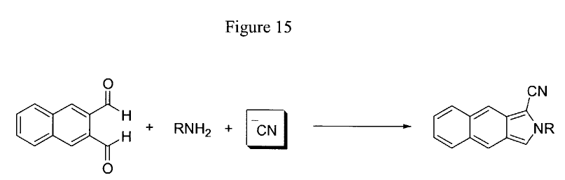

The cyanide concentration may be measured by derivatization of the cyanide with a fluorescing agent such as o-phthalaldehyde (OPA) or other similar compounds to give a fluorescent product. An exemplary derivatization reaction is shown in FIG. 14. Alternatively, naphthalene-2,3-dialdehyde (NDA), anthracene dicarboxyaldehyde (ADA) or equivalents or similar compounds may be used as the fluorescing agent to derivatize the cyanide for determining the cyanide concentration. An example of this reaction is shown in FIG. 15. The OPA may be substituted at one or both of the 4 and 5 positions with substituents, which are capable of enhancing the stability and fluorescence quantum of the isoindole product. For example, methoxy and dimethylamino substituents may be used for one or more of these purposes.

Fluorescing agents similar to o-phthalaldehyde (OPA), anthracene dicarboxyaldehyde (ADA) and/or naphthalene-2,3-dialdehyde (NDA) can be employed in various aspects of the invention, e.g., the methods and kits of the invention, such as

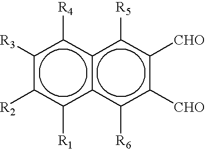

or variations thereof wherein one or more aromatic carbons are substituted with a heteroatom or a hetero group and/or one or both of the CHO groups can be a —COR group, wherein R is selected from the group consisting of an alkyl group, an aryl group or an alkoxy group, wherein the substituents R₁-R₆ are selected from one of the following:

(A) R₁ is selected from —H, an alkyl group, an aryl group, —N(CH₃)₂, —SO₃H, —NO₂, —SO₃⁻Na⁺ and

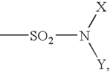

wherein X and Y may be the same or different and are independently selected from —H, an alkyl group, an aryl group and C₁-C₈ alkyl groups, and R₂-R₆ are —H;

(B) R₁, R₄, R₅ and R₆ are —H, an alkyl group or an aryl group, and R₂ and R₃ are combined to form one of:

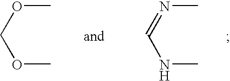

(C) R₁ and R₄ are independently selected from an alkyl group, an aryl group, —N(CH₃)₂ and

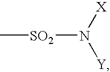

and R₂, R₃, R₅ and R₆ are —H, an alkyl group or an aryl group;

(D) R₁, R₂, R₃, and R₄ are —H, an alkyl group, an aryl group, and R₅ and R₆ are independently selected from an alkyl group, an aryl group, —OCH₃,

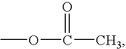

—OSi(CH₃)₂C₄H₉, and N(CH₃)₂; or

(E) R₁, R₄, R₅, and R₆ are —H, and R₂ and R₃ are independently selected from an alkyl group, an aryl group, —CH₃O ,

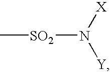

—SO₃H, —CO₂H, and salts thereof; and

(F) R₁, R₃, R₄, R₅ and R₆ are H an alkyl group or an aryl group,and R₂ is —(CH₃)₂N.

As used herein, the term “alkyl” is used to refer to a branched or unbranched, saturated or unsaturated, univalent or bivalent hydrocarbon radical having from 1 to about 50 carbons, 1 to about 40 carbons, or from 1 to about 30 carbons, or, from about 4 to about 20 carbons, or, from about 6 to about 18 carbons, or any variation thereof, including, e.g., aryl, alkene or alkyne groups. When the alkyl group has from 1 to about 6 carbon atoms (e.g., a methyl group, an ethyl group, etc.), it can be referred to as a “lower alkyl.” The term “alkyl” includes alkyl radicals, for example, structures containing one or more methylene, methine and/or methyne groups. The term also includes branched structures have a branching motif similar to i-propyl, t-butyl, i-butyl, 2-ethylpropyl, etc. As used herein, the term encompasses “substituted alkyls.” “Substituted alkyl” refers to an alkyl as just described including one or more functional groups such as lower alkyl, aryl, acyl, halogen (i.e., alkylhalos), hydroxy, amino, alkoxy, alkylamino, acylamino, thioamido, acyloxy, aryloxy, aryloxyalkyl, mercapto, thia, aza, oxo, both saturated and unsaturated cyclic hydrocarbons, heterocycles and the like. These groups may be attached to any carbon of the alkyl moiety. Additionally, these groups may be pendent from, or integral to, the alkyl chain. The term “alkyl”includes arenes, including any substituted or unsubstituted mono- or polycyclic aromatic hydrocarbon compound as well as any mono- or polycyclic heteroaromatic compounds, and can include fused or bridged ring systems.

These fluorescing agents can be used to measure cyanide concentration if they are reacted with a cyanide-containing mixture, when, for example, using a stoichiometric excess of amine. In one aspect, these fluorescing agents, when used in the assay of the present invention, are reacted with a degradation product of the substrate to form a compound that can be detected using a spectrometer, such as a fluorometer, an IR spectrometer, a UV spectrometer, or other suitable, conventional spectrometers for detecting fluorescent materials. Alternatively, the fluorescing agents can be reacted with cyanide starting materials or reaction products, or a product of the reaction can be converted to cyanide for reaction with the fluorescing agent in order to determine conversion due to the hydrolysis reaction.

The fluorescent compound which results from reaction of the fluorescing agent with cyanide can be a compound selected from the group consisting of the following compounds:

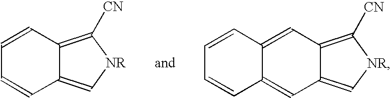

wherein R is an alkyl or aryl group.

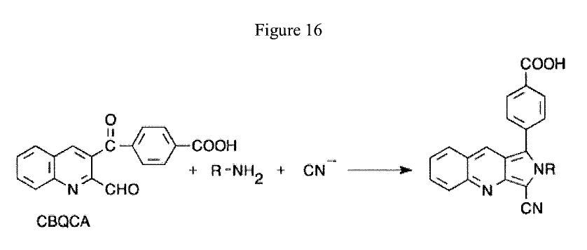

In one aspect, the fluorescing agent comprises a 3-(4-carboxybenzoyl)quinoline-2-carboxaldehyde (CBQCA, ATTO-TAG™) (Molecular Probes, Inc., Eugene, Oreg.) or equivalent. This reagent reacts specifically with amines to form charged conjugates that can be analyzed by electrophoresis techniques. In one aspect, CBQCA reacts with a primary amine and a cyanide group as illustrated in FIG. 16. CBQCA conjugates are maximally excited at ˜456 nm or by the 442 nm spectral line of the He—Cd laser, with peak emission at ˜550 nm. In capillary zone electrophoresis, the sensitivity of amine detection of the laser-induced fluorescence should be in the subattomole range (<10-18 moles) for CBQCA. Sensitivity for detection of reductively aminated glucose using CBQCA is reported to be 75 zeptomoles (75 10-21 moles). CBQCA-derivatized amino acids can also be detected by ultrasensitive detection, e.g., by a capillary electrophoresis, or equivalent.

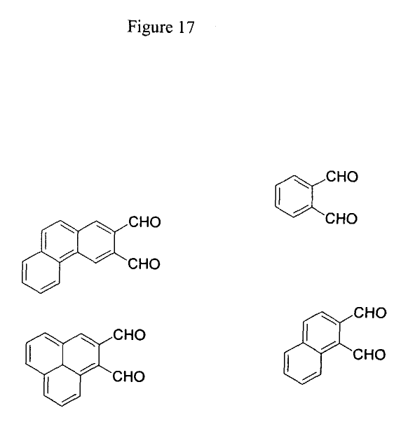

In one aspect, the fluorescing agent comprises structures as set forth in FIG. 17. All of the exemplary fluorescing agents set forth herein can be used in the kits and methods of the invention.

In one aspect, the fluorescing agent is used in the form of a solution. In one aspect, the fluorescing agent is used in the form of a solution with a concentration of about 10 mM to about 1 M. In one aspect, the concentration of the fluorescing agent in solution is about 30 mM to about 100 mM. In one aspect, the solution of fluorescing agent further comprises a buffer to control the pH. Any suitable buffer can be used as long as it controls the pH properly. In one aspect, the pH range of the solution of fluorescing agent is between about 8.5 and about 12.5. In one aspect, the pH range of the solution is between about 10.4 and about 12.5.

In one aspect, in order to determine cyanide concentration, the sample containing the cyanide is added to a fresh, buffered pH-controlled solution (pH 7-10), for example, of about 1 to about 500 millimolar aromatic dicarboxaldehyde, or, about 1 to about 1000 millimolar primary amine in a buffered pH-controlled solution (pH 7-10) at a temperature, e.g., ranging from about 18° C. to 40° C. After the reaction has gone substantially to completion, generally in about 10 seconds to 1 week, or, in about 10 minutes to one hour, the concentration of cyanide can be determined by measuring the amounts of the adducts in the solution using high performance liquid chromatography with fluorescence or chemi-luminescence detection. In alternative aspects, the optimal wavelengths for excitation of the products produced from OPA are 230 nm and about 320-340 nm, and the optimal wavelength for emission is about 375-385 nm. With respect to adducts produced from the NDA compounds, the optimal excitation wavelengths can be about 250 nm, 420 nm and 450 nm whereas the optimal emission wavelength is about 490 nm. Optional wavelengths may vary slightly for substituted OPA, ADA and NDA fluorescing agents.

Suitable amines for use in the methods and kits of the present invention include alkylamines, arylamines and amino acids. In alternative aspects, primary amines or amino acids are employed. Exemplary amines useful in the present invention include glycine, alanine, tyrosine, valine, phenylalanine, aspartic acid, glutamic acid, cysteic acid. serine, histidine, threonine, isoleucine, methionine, tryptophan, arginine, asparagines, GABA, n-acetyl lysine, and glutamine.

The kits and assays of the present invention can be used as a screening technique to screen for a particular enzymatic or catalytic activity. The particular activity of interest in the present invention may be the activity of a catalyst, which catalyzes, for example, the hydrolysis of nitrile groups in nitrile-group containing compounds. In this case, the catalyst is employed in a hydrolysis reaction, the reaction is quenched and the amount of nitrile-group containing compound remaining in the reaction mixture is then determined as described above.

The present invention may also be employed as an assay method for detecting if a biological sample has a particular activity, such as for enzymatic hydrolysis of nitrile-group containing compounds. Such a method may comprise the steps of: contacting a biological sample with a suitable nitrile group-containing substrate in the presence of water to cause hydrolysis of at least some of the nitrile-groups in the substrate, quenching the reaction to a pH of about 10 to about 12, or, about pH 11 to about 11, to stop the hydrolysis reaction and decompose at least a portion of the remaining nitrile-group containing compound to produce cyanide, contacting the cyanide-containing mixture with a fluorescing agent such as those specified above, for a suitable period of time to form a fluorescent compound; detecting the concentration of the fluorescent compound, and calculating the concentration of the nitrile group-containing substrate remaining in the reaction mixture to determine if the biological sample has the desired activity.

In one aspect, the final step of the assay method of the present invention further involves: measuring the fluorescence intensity emitted from the fluorescent compound; comparing the measured fluorescence, the concentration of cyanide in the sample, and determining the activity of the biological sample based on the amount of cyanide in the sample by relating the amount of cyanide to the degree of conversion of the nitrile-group containing starting material. Alternatively negative control samples or positive control samples can be assayed along with the actual samples to provide baselines for determining which biological samples have the desired activity.

The biological sample used in the present invention can be derived from a wide range of sources including, for example, environmental samples containing more than one organism (e.g., mixed populations of organisms), enriched samples, samples from an isolated organism (cultured or uncultured), and the like. The biological sample may also include microorganisms existing in nature, microorganisms isolated from nature, microorganisms from any type of library, clones from any library, enzymes, materials containing an enzyme, cells, DNA molecules, RNA molecules, or suitable living organisms. In one aspect, the biological sample used in the present invention is selected from microorganisms, whole cells, enzymes and clones that contain samples from mixed population libraries. In one aspect, the biological sample is in the form of a whole cell suspension or contains a clone from a mixed population library. The library may be a library derived from more than one organism, such as a mixed population of organisms from, for example, an environmental sample or an uncultivated population of organisms or a cultivated population of organisms.

“Mixed population libraries”can be generated from samples containing one or more microorganism and represent partial or entire genomes of these organisms. The DNA from the samples can be archived in cloning vectors that can be propagated in suitable hosts. The library can be produced from DNA which is recovered without culturing of an organism, particularly where the DNA is recovered from an environmental sample containing microorganisms which are not or cannot be cultured. See, e.g., U.S. Pat. Nos. 6,280,926; 5,958,672. Optionally, normalization (for example, as described in U.S. Pat. Nos. 6,001,574 and 5,763,239) of the DNA present in these samples could be performed, allowing a different representation of the DNA from the species than the representation of the DNA from the species present in the original sample. This can dramatically increase the efficiency of finding interesting genes from minor constituents of the sample, which may be under-represented by several orders of magnitude compared to the dominant species.

A whole cell suspension, such as an E. Coli suspension, is well known to a person skilled in the art. The biological sample produces or includes an enzymatic or other type of catalyst for the reaction in question. The assay of the present invention can be employed identify biological samples with activity as a catalyst for the reaction in question.

In one aspect, the present invention is employed in a high throughput screening method such as those employing microarrays, e.g., GIGAMATRIX™ (Diversa Corporation, San Diego, Calif.), as described, e.g., in PCT publication WO 01/38583, and/or FACS (for example, as described in U.S. Pat. No. 6,174,673 to Short et al.). For example, in one high throughput method, the reaction can be carried out in small wells in plates, such as 96-well plates, 384-well plates and 1536-well plates, which are widely used in the biotechnology industry for sample screening purposes.

In one aspect, the particular activity of interest in the present invention is an enzymatic activity. In one aspect, the particular activity of interest is an enzymatic activity that catalyzes the hydrolysis of nitrile groups in a nitrile-group containing molecule. In one aspect, the particular activity of interest in the present invention is an enzymatic activity that catalyzes the hydrolysis of a compound selected from a-hydroxynitriles and aminonitriles. In one aspect, the particular activity of interest in the present invention is the activity of an enzyme selected from nitrilases, nitrile hydratases and hydroxynitrile lyases (oxynitrilases).

Some of the nitrilases that can be screened for nitrilase activity on various substrates using the assay of the present invention are listed in the “Sequence Listing” of the invention. Those nitrilases can have sequences as set forth in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384 and/or 386, or, nitrilases having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91% 92%, 93%, 94%, 95%, 96%, 97%, 98%, 99%, or more sequence identity to one of these sequences. The sequence identities can be determined by analysis with a sequence comparison algorithm or by a visual inspection. The sequence comparison algorithm can be a BLAST version 2.2.2 algorithm where a filtering setting is set to blastall -p blastp -d “nr pataa” -F F, and all other options are set to default.

Alternatively, those nitrilases may also be encoded with one or more DNA sequences selected from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 374, 381, 383, and/or 385.

The fluorescent compound may be measured using, for example, a fluorometer, or an equivalent instrument, to detect fluorescence, including, e.g., fluorescence polarization, time-resolved fluorescence or FRET. In addition, FACS, a chromatographic technique (e.g., a liquid chromatograph, such as HPLC) or capillary electrophoresis (CE) techniques can be used. In general, excitation radiation, from an excitation source having a first wavelength, causes the excitation radiation to excite the sample. In response, fluorescence compounds in the sample emit radiation having a wavelength that is different from the excitation wavelength. Methods of performing assays on fluorescent materials are well known in the art and are described, e.g., by Lakowicz (Principles of Fluorescence Spectroscopy, New York, Plenum Press, 1983) and Herman (“Resonance energy transfer microscopy,” in: Fluorescence Microscopy of Living Cells in Culture, Part B, Methods in Cell Biology, vol. 30, ed. Taylor & Wang, San Diego, Academic Press, 1989, pp. 219-243).

In addition, fluorescence activated cell sorting (FACS) can be used to screen and isolate clones having an activity or sequence of interest. FACS machines have been employed in studies focused on the analyses of eukaryotic and prokaryotic cell lines and cell culture processes.

In one aspect, the present invention has an advantage that it does not require cells to survive, as do previously described technologies, since the desired enzyme nucleic acid (recombinant clones) can be obtained from live or dead cells. The cells only need to be viable long enough to produce the compound to be detected, and can thereafter be either viable or non-viable cells so long as the expressed biomolecule remains active. The present invention also solves problems that would have been associated with detection and sorting of E. coli expressing recombinant enzymes, and recovering encoding nucleic acids.

The method and kits of the present invention can be implemented using a high throughput screening methodology, e.g., by using robotic technology in combination with high-density plates. A person skilled in the art can employ the above described assay method in a high throughput screening assay using common general knowledge and the above-teachings.

In addition, the assays and kits of the present invention may also be employed as an assay for screening genes encoding enzymes with a particular enzymatic activity by adding a step of expressing the genes in a suitable environment, such as in a suitable vector.

In order to quantitatively determine the activity of a particular biological sample or enzyme in a high-throughput screening process, the residual substrate concentration of the nitrile-group containing compound in a well of a plate is measured. In order to derive the concentration of the substrate from the fluorescence reading, calibration measurements for different substrates have been carried out. Calibration measurements may be carried out by the following procedure:

1) Add a known concentration of a particular nitrile solution (10 μL) to a plate containing 50 μL of the OPA reagent described in Example 3;

2) let the plate sit for 5 minutes;

3) add 40 μL of glycine solution (prepared according to the procedure in Example 3, 625 mM) to the plate;

4) allow the plate to sit for 20 minutes; and

5) determine the fluorescence of the sample (excitation=330 nm, emission=380 nm).

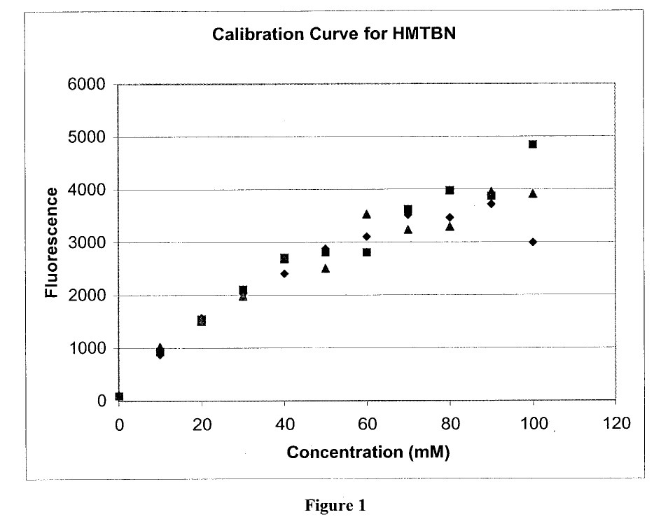

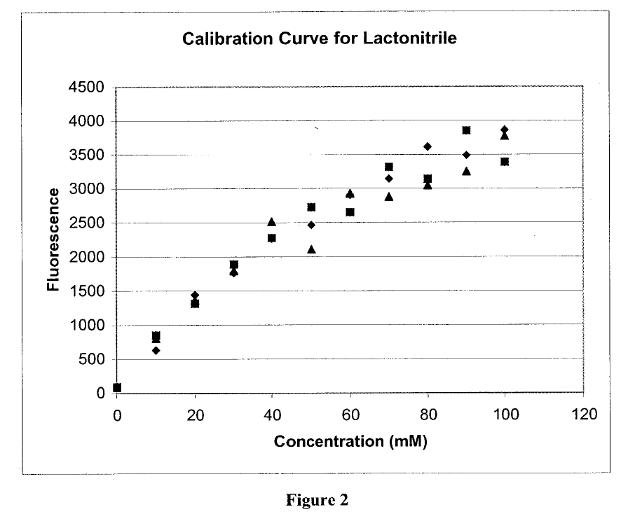

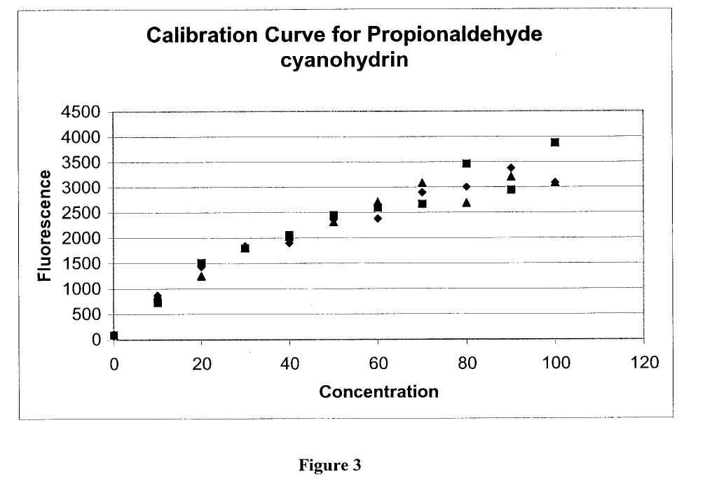

Calibration curves plotting fluorescence readings against substrate concentration have been plotted for various substrates and are illustrated in FIGS. 1-3. FIGS. 1-3 illustrate the calibration curves (or standard curves) using HMTBN, lactonitrile and propionaldehyde cyanohydrin as the substrates, respectively. The calibration curves in FIGS. 1-3 also demonstrate the assay sensitivity and illustrate the broad substrate scope of the present invention.

### EXAMPLES

### Example 1

Assay Development and Validation

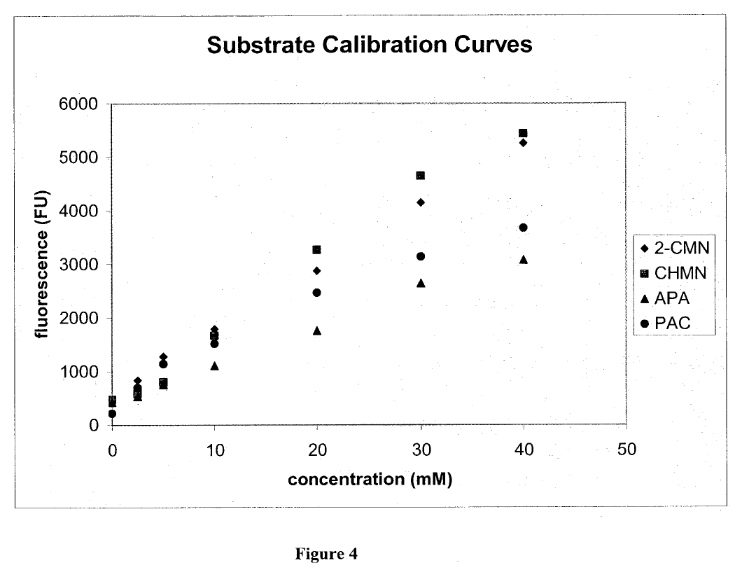

Calibration curves were established using OPA as the fluorescing agent for the following substrates: 2-chloromandelonitrile (CMN) with an R² value of 0.998, cyclohexylmandelonitrile (CHMN), with an R₂ value of 0.99, acetophenone aminonitrile (APA) with an R² value of 0.99, and phenylacetaldehyde cyanohydrin (PAC) with an R² value of 0.97 as shown in FIG. 4.

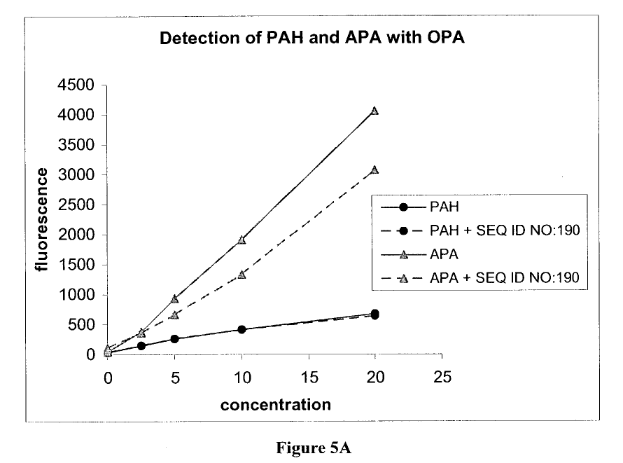

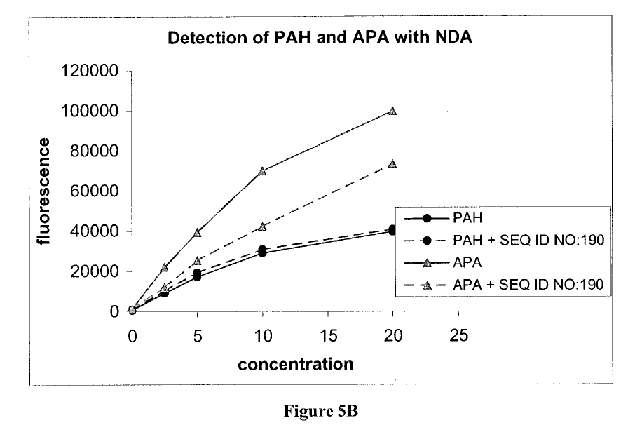

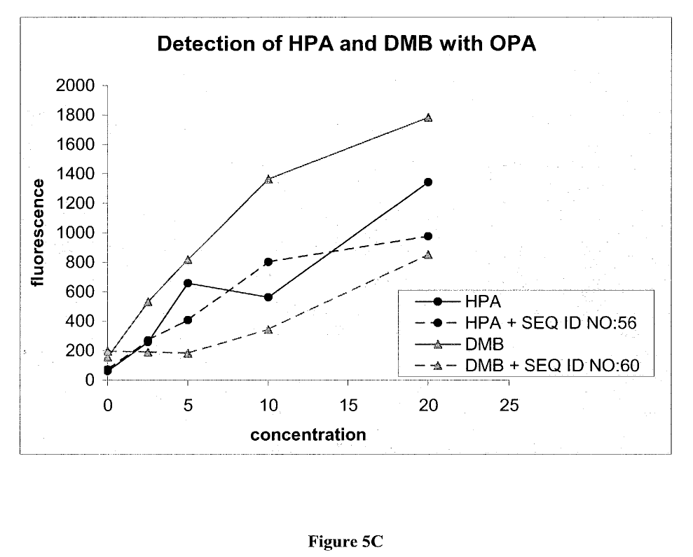

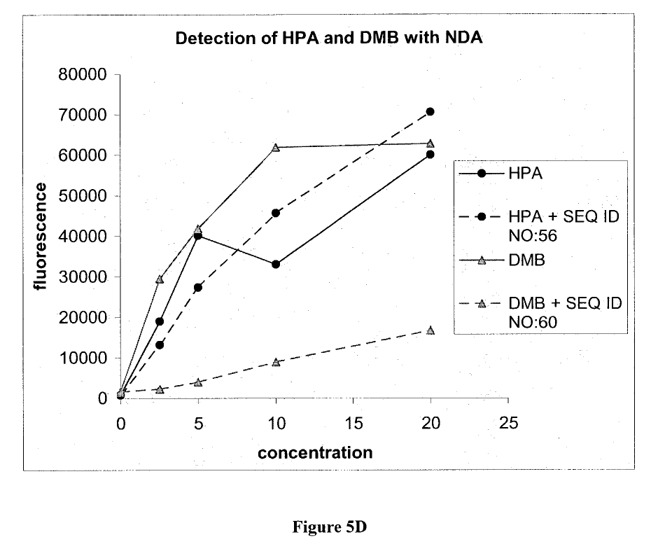

In assays using certain substrates, naphthalene dicarboxaldehyde (NDA) was substituted for OPA as the fluorescing agent. Calibration curves for APA, PAH, HPA and DMB, with either OPA or NDA were constructed (see FIGS. 5A, 5B, 5C and 5D). To determine sensitivity and background fluorescence, lyophilized nitrilase lysates with SEQ ID NOS: 190, 56, and 60 were added, respectively. Hydrolysis was detected when APA, HPA or DMB was used as the enzyme substrate. As shown in FIGS. 5A, 5B, 5C and 5D, NDA sharply boosted the signal, often by an order of magnitude of the assay of the present invention in comparison a similar assay using OPA. The reduced linearity in the standard curves is mostly likely caused by signal saturation (see FIGS. 5B and 5D).

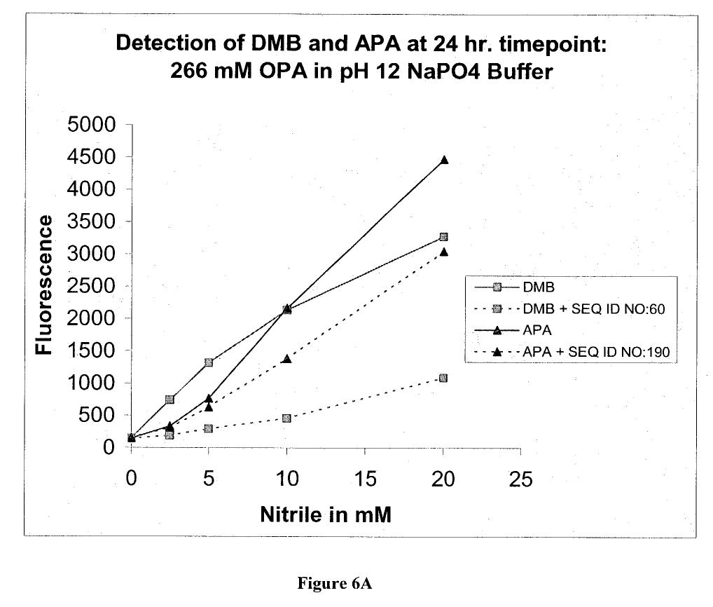

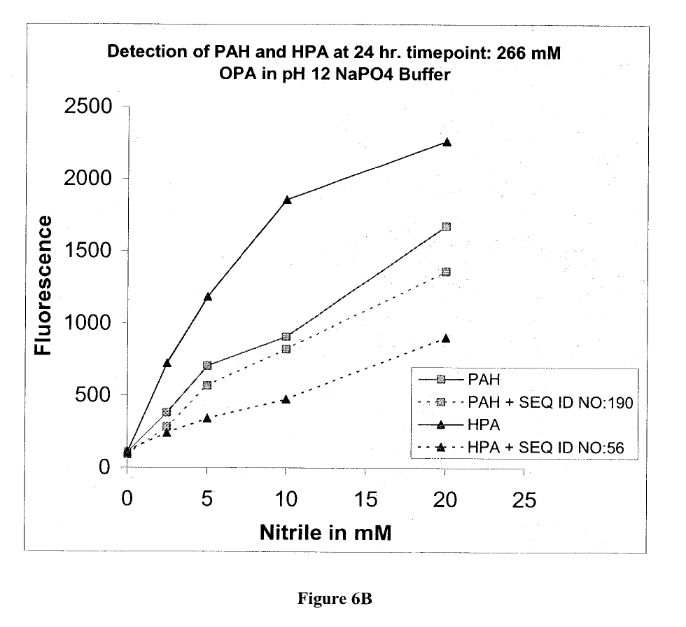

NDA was established as an alternative detection reagent for aliphatic compounds. However, it is desirable for an assay of the present invention to utilize the same detection system for all of the substrates since this would facilitate the automated evaluation of multiple nitrilase substrates and/or multiple nitrilases. The OPA based assay of the present invention is clearly effective for the analysis of aromatic substrates PAC, CMN, CHMN, APA, MN and PGN. In addition, acceptable OPA assays have been verified for the aliphatic substrates PAH, HPA and DMB. Calibration curves of the OPA based assay for the aliphatic substrates PAH, HPA, and DMB using a lyophilized nitrilase lysate with known catalytic activity for each of the substrates are shown in FIGS. 6A and 6B. As shown in FIGS. 6A and 6B, the effectiveness of the OPA assay in a pH 12 buffer is improved over an OPA assay in a PH 10 (shown in FIGS. 5A and 5C) buffer for substrates such as PAH, HPA and DMB. Therefore, the OPA-based assay of the present invention has the further advantage that it can be used for a wide variety of substrates without significantly modifying the detection system. Accordingly, it is advantageous to implement the assay of the present invention in a high throughput automated system using a variety of substrates.

### Example 2

Whole Cell Optimization

The OPA assay of the present invention was further evaluated and optimized for nitrilase activity detection in a whole cell format. Both nitrilase expressing whole cells and in-situ lysed cells were evaluated. Lyophilized cell lysates were evaluated in comparison to their respective whole cell clones as controls. Mandelonitrile (MN) was employed as the substrate.

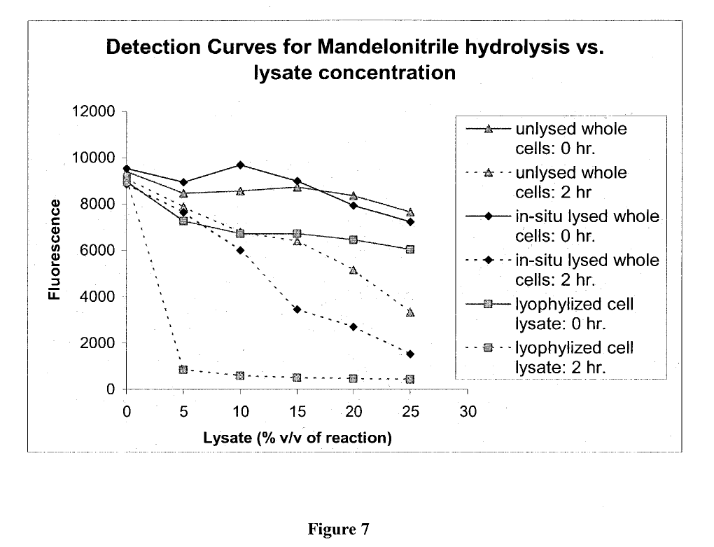

The lyophilized cell lysate, including a nitrilase having SEQ ID NO: 188, was evaluated in comparison to whole cells expressing the nitrilase having a SEQ ID NO: 188 and in situ lysed cells expressing the nitrilase having a SEQ ID NO: 188. The addition of whole cells did not affect fluorescence nor result in fluorescence quenching. Three cell lysing solutions including B-PER (Pierce Chemical Company, Rockford, Ill.), BUGBUSTER™ (Novagen, Madison, Wis.) and CELLYTIC B-II (Sigma, St. Louis, Mo.) were evaluated and were found not to have a deleterious affect on the OPA assay. Addition of any of the three cell lysis solutions improved permeability (and therefore conversion) of mandelonitrile in the whole cell systems. Also, the addition of a product of the hydrolysis reaction used in the assay of the present invention, such as α-hydroxyacid or α-aminoacid, did not affect the detection sensitivity by the OPA assay. FIG. 7 illustrates the results of the hydrolysis of mandelonitrile with whole cells expressing a nitrilase having SEQ ID NO: 188 (“unlysed whole cells”), the cells lysed with B-PER (“In-situ lysed whole cells”) and the cell lysate after having been further lyophilized (“lyophilized cell lysate”).

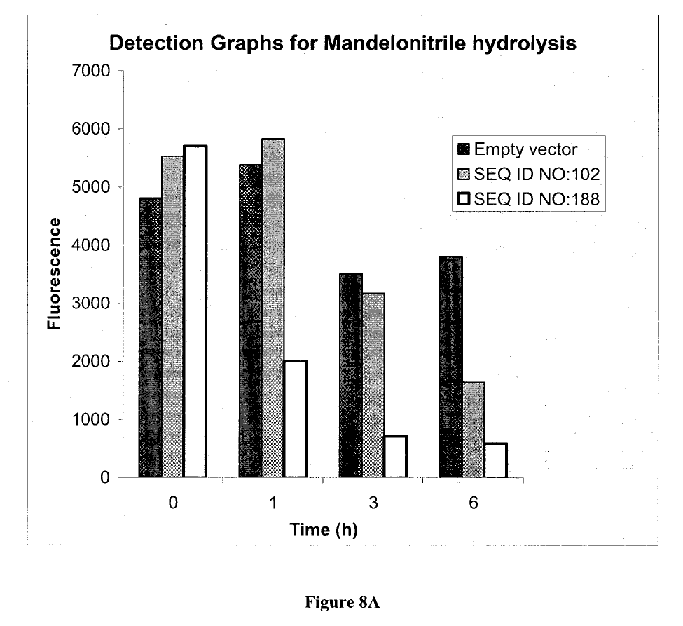

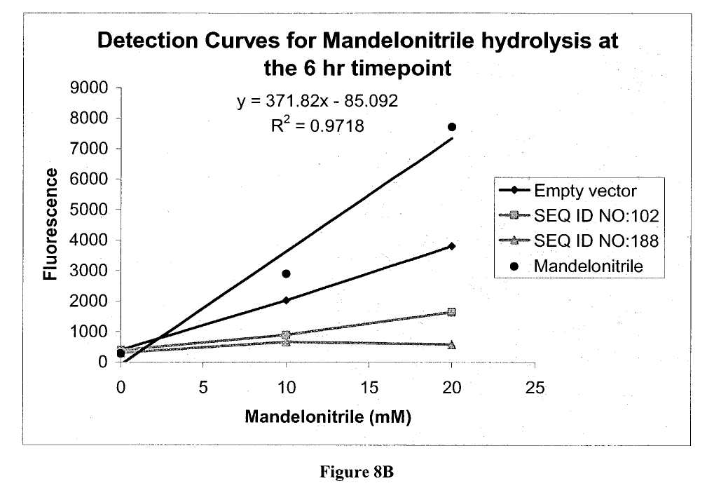

The assay was further modified from its original format, which required several liquid transfer steps, into a one plate process, where cell growth, nitrile hydrolysis and the OPA derivatizing reaction were carried out in the same microtiter plate. Mandelonitrile was tested using this single well format. In this case, an E. coli. gene site-saturation mutagenesis (GSSM™) cell host was used as the host cell for expressing a nitrilase. Three clones encoding a nitrilase having SEQ ID NOS: 102, and 188, and an empty vector, respectively, were tested. The clone containing the empty vector was used as a control. Hydrolysis was evaluated at four timepoints, at 10 and 20 mM, and also with a 0 mM control (FIGS. 8A and 8B).

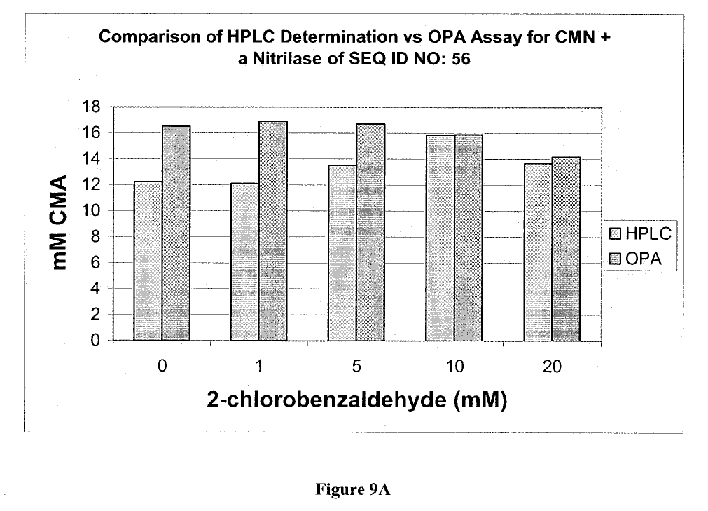

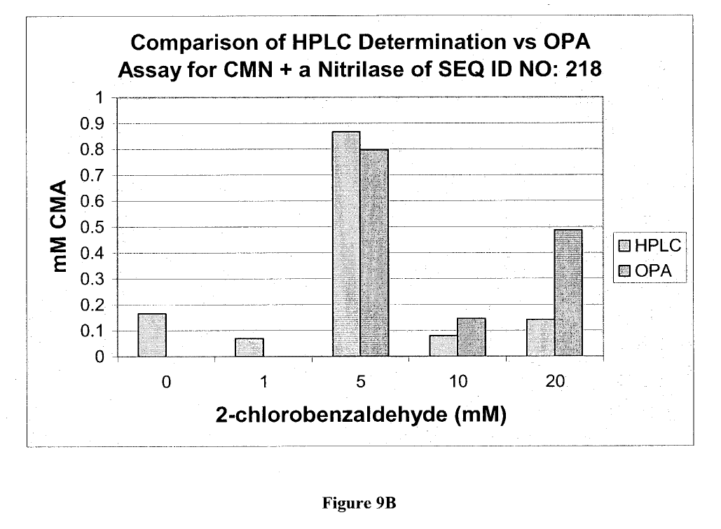

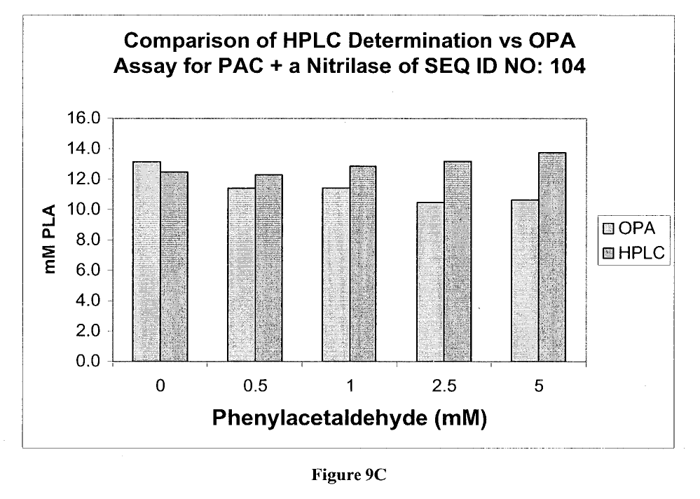

To further verify the effectiveness of the assay of the present invention, experiments were performed to assess the inhibition of chlorobenzaldehyde and phenylacetaldehyde using lyophilized cell lysate containing various enzymes. The experiments were monitored by both HPLC and an OPA assay of the present invention. A comparison of the conversion calculated by the OPA assay versus that determined by HPLC for the CMN and PAC substrates showed good agreement between these two methods as shown in FIGS. 9A, 9B and 9C. FIG. 9A shows the two hour timepoint of an enzyme having SEQ ID NO: 56 on 2-chloromandelonitrile, wherein CMA stands for chloromandelic acid. FIG. 9B shows the same timepoint of an enzyme having a SEQ ID NO: 218 on chloromandelonitrile, wherein CMA stands for chloromandelic acid. FIG. 9C shows the two hour timepoint of an enzyme having SEQ ID NO: 104 on phenylacetaldehyde cyanohydrin, wherein PLA stands for phenyl lactic acid.

### EXAMPLE 3:

Exemplary Nitrilase Screening Method

The following is a representative example of a procedure of the invention for screening an expression library for nitrilase activity using 384-well plates.

Preparation of Reagents:

500 ml of OPA reagent is prepared by mixing 125 mL of methanolic OPA solution (266 mM) with 375 mL of borate buffer (1 M, pH 10.0).

1 L of a primary amine-containing solution (glycine solution, 625 mM) is prepared by adding 147 ml of 4.25 M glycine stock to 853 ml of water.

The substrate solution (reaction media) is prepared by mixing equal amounts of 111 mM phosphate (pH 7.0) and 111 mM cyanohydrin.

Assay Procedure:

To each well, 10 μL of whole cells from a clone of an expression library was added. This process was repeated for each well so that each well contained a different clone from the expression library. Then 90 μl of reaction media was added to each well to form the reaction mixture.

10 μL of the reaction mixture from each well was (robotically) transferred to a new well containing 50 μL of the OPA reagent, thus adjusting the pH to 10 and quenching the enzymatic reaction. The samples were allowed to incubate for 5 minutes at 37° C. 40 μL of the glycine solution was added using titertek. The samples were allowed to incubate for another 20 minutes at 37° C. Then, the fluorescence value for each sample was recorded (excitation=330 nm, emission=380 nm) on a plate reading fluorometer. The data indicates the degree of conversion of the nitrile by the hydrolysis reaction and from this the nitrilase activity of each clone can be determined.

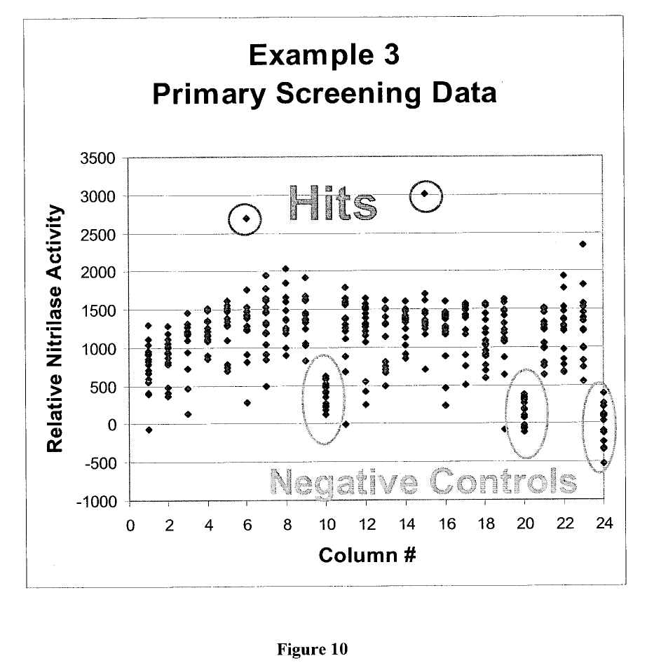

Screening results obtained using clones from an expression library are illustrated in FIG. 10. During this screening process, negative control biological samples were employed to verify the procedure. Typically, the negative control samples have little or no nitrilase activity. The negative controls are illustrated in columns 10, 20 and 24 in FIG. 10. In FIG. 10, two hits (clones with positive nitrilase activity above the desired minimum activity threshold for this screening process) that were identified in this primary nitrilase screening process are illustrated.

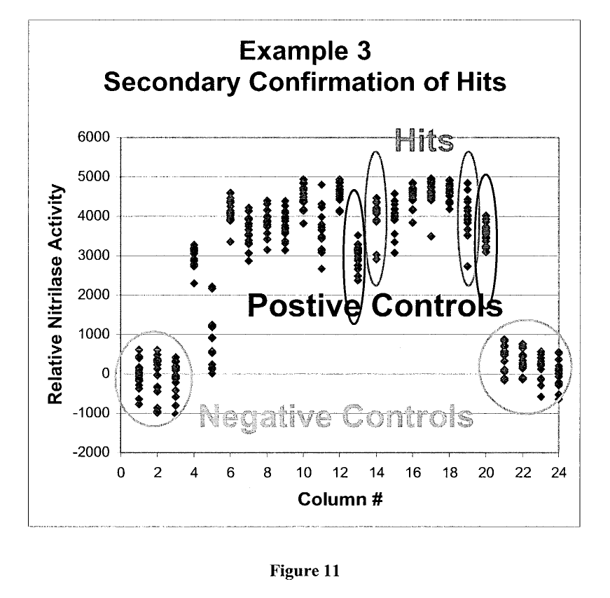

In addition to this type of primary screening, the hits obtained in the primary screening may be further tested or screened using the same procedure in a secondary confirmation screening. In an exemplary secondary confirmation screening, both negative control biological samples and positive control biological control samples were screened side-by-side with the clones (or hits) to be screened. Typically, the positive control samples have a particular level of known nitrilase activity which can be used as a baseline for identifying good nitrilase candidates. The results of a typical secondary confirmation screening are illustrated in FIG. 11. During the secondary screening of the hits, each clone was loaded into a column (14 or 19) on the assay plate. The negative controls were in columns 1-3 and 21-24. The positive controls (wild type) were in columns 13 and 20. Columns 6 through 12 and 15 through 18 were confirmations of other hits. The secondary confirmation screening depicted in FIG. 11 confirmed the activity of the hits identified by the primary screening depicted in FIG. 10.

The foregoing examples were presented for the purpose of illustration and description only and are not to be construed as limiting the invention in any way.

A number of embodiments of the invention have been described. Nevertheless, it will be understood that various modifications may be made without departing from the spirit and scope of the invention. Accordingly, other embodiments are within the scope of the following claims.

What is claimed is:

1. A method for monitoring a chemical or biochemical process, comprising the following steps:
(a) providing a reactant comprising a cyanide or a material that can be converted to a cyanide, or, a reactant that generates as a reaction product a cyanide or a material that can be converted to cyanide;
(b) reacting the reactant and monitoring the reaction by sampling the reactant or a product and, if the reactant is a material that can be converted to a cyanide or the reactant generates a material that can be converted to cyanide, converting the reactant or the product to a cyanide; and
(c) measuring the cyanide concentration in the sample, thereby monitoring the chemical or biochemical process.

2. A method for assaying catalytic activity of a material in a chemical or biochemical process, or a material in a biological sample, comprising the following steps:
(a) providing a reactant comprising a cyanide or a material that can be converted to a cyanide, or, a reactant that generates as a reaction product a cyanide or a material that can be converted to cyanide;
(b) reacting the reactant and monitoring the reaction by sampling the reactant or a product and, if the reactant is a material that can be converted to a cyanide or the reactant generates a material that can be converted to cyanide, converting the reactant or the product to a cyanide; and
(c) measuring cyanide concentration in the sample and determining the degree of conversion of the reaction from the cyanide concentration, thereby assaying the catalytic activity of the material during the reaction.

3. The method of claim 1 or claim 2, wherein the cyanide concentration in the sample is measured by using a chemical technique.

4. The method of claim 1 or claim 2, wherein the cyanide concentration is measured by derivatization of the cyanide with a fluorescing agent.

5. The method of claim 4, wherein the cyanide concentration in the sample is measured by using a fluorescence detection technique.

6. The method of claim 4, wherein the fluorescing agent comprises a naphthalene-2,3-dialdehyde (NDA) or equivalent or anthracene dicarboxyaldehyde (ADA) or equivalent.

7. The method of claim 4, wherein the fluorescing agent comprises an o-phthalaldehyde (OPA), a structure as set forth in FIG. 17, or an equivalent structure.

8. The method of claim 7, wherein the o-phthalaldehyde (OPA) is substituted at one or both of the 4 and 5 positions with substituents capable of enhancing the stability and fluorescence quantum of an isoindole product.

9. The method of claim 8, wherein the substituents comprise a methoxy substituent, a dimethyl amino substituent or both.

10. The method of claim 4, wherein the fluorescing agent comprises a composition having a formula comprising 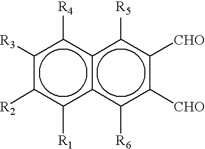
or variations thereof wherein one or more aromatic carbons are substituted with a heteroatom or a hetero group and/or one or both of the CHO groups can be a —COR group, wherein R is selected from the group consisting of an alkyl group, an aryl group or an alkoxy group,
wherein the substituents R₁, R₂, R₃, R₄, R₅, R₆ are selected from one of the following:
(A) R₁ is selected from —H, an alkyl group, an aryl group, —N(CH₃)₂, —SO₃H, —NO₂, —SO₃⁻Na⁺ and 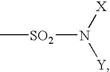
wherein X and Y may be the same or different and are independently selected from —H, an alkyl group, an aryl group and C₁-C₈ alkyl groups, and R₂-R₆ are —H;
(B) R₁, R₄, R₅ and R₆ are —H, an alkyl group or an aryl group, and R₂ and R₃ are combined to form one of: 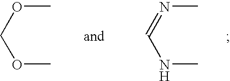
(C) R₁ and R₄ are independently selected from an alkyl group, an aryl group, —N(CH₃)₂ and 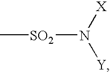
and R₂, R₃, R₅ and R₆ are —H, an alkyl group or an aryl group;
(D) R₁, R₂, R₃, and R₄ are —H, an alkyl group, an aryl group, and R₅ and R₆ are independently selected from an alkyl group, an aryl group, —OCH₃, 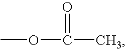
—OSi(CH₃)₂C₄H₉, and N(CH₃)₂; or
(E) R₁, R₄, R₅, and R₆ are —H, and R₂ and R₃ are independently selected from an alkyl group, an aryl group, —CH₃O, 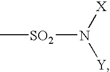
—SO₃H, —CO₂H, and salts thereof; and
(F) R₁, R₃, R₄, R₅ and R₆ are H an alkyl group or an aryl group, and R₂ is —(CH₃)₂N.

11. The method of claim 10, wherein the fluorescing agent is reacted with a cyanide-containing mixture using a stoichiometric excess of amine and used to measure cyanide concentration.

12. The method of claim 10, wherein the heteroatom or hetero group comprises a nitrogen, an oxygen, a sulfur, a mercapto group, a thia group, a thio group, an aza group or an oxo group.

13. The method of claim 4, wherein the fluorescing agent is reacted with a degradation product of a substrate to form a compound that can be detected using a spectrometer.

14. The method of claim 13, wherein the spectrometer is a fluorometer, an IR spectrometer or a UV spectrometer.

15. The method of claim 4, wherein the fluorescing agent is reacted with a cyanide starting material or a reaction product, or a product of the reaction can be converted to cyanide for reaction with the fluorescing agent, to determine conversion due to a hydrolysis reaction.

16. The method of claim 4, wherein the fluorescing agent comprises a compound selected from the group consisting of: 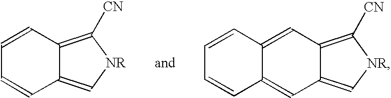
wherein R is an alkyl or aryl group.

17. The method of claim 4, wherein the fluorescing agent comprises a 3-(4-carboxybenzoyl)quinoline-2-carboxaldehyde (CBQCA, ATTO-TAG™).

18. The method of claim 17, wherein the optimal wavelength for excitation of products produced from 3-(4-carboxybenzoyl)quinoline-2-carboxaldehyde (CBQCA) or equivalent is about 488 nm, and the optimal wavelength for emission is about 570 nm.

19. The method of claim 17, wherein the 3-(4-carboxybenzoyl)quinoline-2-carboxaldehyde (CBQCA) or equivalent is used in solution at a concentration of between about 10⁻¹⁵mole/liter (M) to about 1 mole/liter (M).

20. The method of claim 1 or claim 2, wherein the reaction is quenched to produce a cyanide-containing reaction mixture.

21. The method of claim 1 or claim 2, wherein the reactant comprises a substrate for a reaction.

22. The method of claim 21, wherein the substrate comprises a cyanide, a nitrile-group containing compound or a mixture thereof.

23. The method of claim 21, wherein the substrate comprises an α-hydroxynitrile, an aminonitrile and/or mixtures thereof.

24. The method of claim 21, wherein the substrate comprises a hydroxymethyl thiobutyronitrile (HMTBN), a lactonitrile, a propionaldehyde cyanohydrin (PAC), a 2-chloromandelonitrile (CMN), a cyclohexylmandelonitrile (CHMN), an acetophenone aminonitrile (APA), a phenylglycine (PGN), a dimethylbutanal aminonitrile (DMB), a hydroxylpivaldehyde aminonitrile (HPA), a pivaldehyde aminonitrile (PAH), mandelonitrile (MN) and/or mixtures of two or more of these compounds.

25. The method of claim 21, wherein the substrate undergoes reactions with high rate constants or reactions which favor relatively high conversion to cyanide when the conversion of a substrate to cyanide involves an equilibrium reaction.

26. The method of claim 21, wherein the reaction is under alkaline conditions to quench chemical hydrolysis of the substrate.

27. The method of claim 21, wherein the substrate is used as a solution with a concentration of between about 10⁻¹⁵ mole/liter (M) to about 10 mole/liter (M).

28. The method of claim 27, wherein the substrate is used as a solution with a concentration of between about 1 μM to about 1 mole/liter (M).

29. The method of claim 28, wherein the substrate is used as a solution with a concentration of between about 1 μM to about 100 μM.

30. The method of claim 27, wherein the substrate is used as a solution with a concentration of between about 100 μM to about 100 mM.

31. The method of claim 27, wherein the substrate is used as a solution with a concentration of between about 10 mM to about 500 mM.

32. The method of claim 31, wherein the substrate is in an aqueous solution at a substrate concentration of between about 30 mM to about 150 mM.

33. The method of claim 4, wherein the fluorescing agent is used in the form of a solution.

34. The method of claim 33, wherein the fluorescing agent is used in the form of a solution with a concentration of between about 10⁻¹⁵ mole/liter (M) to about 1 mole/liter (M).

35. The method of claim 34, wherein the fluorescing agent is used in the form of a solution with a concentration of between about 1 μM to about 500 mM.

36. The method of claim 35, wherein the fluorescing agent is used in the form of a solution with a concentration of between about 1 μM to about 100 mM.

37. The method of claim 36, wherein the concentration of the fluorescing agent in solution is about 30 mM to about 100 mM.

38. The method of claim 33, wherein the fluorescing agent further comprises a buffer to control pH.

39. The method of claim 33, wherein the pH range of the solution of fluorescing agent is between about pH 8.5 and about pH 12.5.

40. The method of claim 39, wherein the pH range of the solution is between about pH 10 and about pH 11.

41. The method of claim 1 or claim 2, wherein in order to determine cyanide concentration a sample containing cyanide is added to a fresh, buffered pH-controlled solution of about pH 7 to about 10.

42. The method of claim 38, wherein the buffered pH-controlled solution comprises about 1 to about 500 millimolar aromatic dicarboxaldehyde.

43. The method of claim 38, wherein the buffered pH-controlled solution comprises about 1 to about 1000 millimolar primary amine.

44. The method of claim 38, wherein the primary amine is in a buffered pH-controlled solution of about pH 7 to about 10 at a temperature ranging from about 25° C. to about 40° C.

45. The method of claim 1 or claim 2, wherein the reaction is allowed to continue for about 10 seconds to about 1 week, or, for about 10 minutes to about one hour.

46. The method of claim 1 or claim 2, wherein after the reaction has gone substantially to completion the concentration of cyanide is determined by measuring amounts of adducts in the solution using high performance liquid chromatography (HPLC) with a fluorescence or a chemi-luminescence detection technique.

47. The method of claim 7, wherein the optimal wavelengths for excitation of the products produced from o-phthalaldehyde (OPA) or equivalent are 230 nm and about is 320-340 nm, and the optimal wavelength for emission is about 375-385 nm.

48. The method of claim 6, wherein the optimal wavelengths for excitation of the products produced from naphthalene-2,3-dialdehyde (NDA) or equivalent are about 250 nm, 420 nm or 450 nm and the optimal emission wavelength is about 490 nm.

49. A method for screening a biological sample for a particular activity, comprising the following steps:
(a) providing a substrate, a derivatizing agent and an amine;
(b) providing a biological sample;
(c) combining the biological sample with the substrate to form a reaction mixture, thereby conducting a reaction in the reaction mixture;
(d) contacting the resultant reaction mixture with the derivatizing agent and the amine for a suitable period of time to form a fluorescent compound; and
(e) detecting the fluorescence of the fluorescent compound to determine the activity of interest in the biological sample.

50. The method of claim 49, wherein the amines comprises a primary amine or an amino acid.

51. The method of claim 50, wherein the amine comprises an alkylamine, an arylamines.

52. The method of claim 50, wherein the amine or amino acid comprises a glycine, an alanine, a tyrosine, a valine, a phenylalanine, an aspartic acid, a glutamic acid, a cysteic acid, a serine, a histidine, a threonine, an isoleucine, a methionine, a tryptophan, an arginine, an asparagine, a GABA, an n-acetyl lysine or a glutamine.

53. The method of claim 1 or claim 2, wherein the method is used as a screening technique to screen for a particular enzymatic or catalytic activity.

54. The method of claim 53, wherein the activity of interest is an activity of a catalyst which catalyzes the hydrolysis of nitrile groups in nitrile-group containing compounds.

55. The method of claim 54, wherein the catalyst is employed in a hydrolysis reaction and the reaction is quenched and the amount of nitrile-group containing compound remaining in the reaction mixture is determined.

56. The method of claim 2, wherein the activity comprises enzymatic hydrolysis of nitrile-group containing compounds.

57. The method of claim 56 comprising the steps of: contacting a biological sample with a suitable nitrile group-containing substrate in the presence of water to cause hydrolysis of at least some of the nitrile-groups in the substrate, quenching the reaction to a pH of about 10 to about 12, or, about pH 10 to about 11, to stop the hydrolysis reaction and decompose at least a portion of the remaining nitrile-group containing compound to produce cyanide, contacting the cyanide-containing mixture with a fluorescing agent for a suitable period of time to form a fluorescent compound; detecting the concentration of the fluorescent compound, and calculating the concentration of the nitrile group-containing substrate remaining in the reaction mixture to determine if the biological sample has the desired activity.

58. The method of claim 57, wherein the final step of the method further comprises measuring the fluorescence intensity emitted from a fluorescent compound; comparing the measured fluorescence, the concentration of cyanide in the sample, and determining the activity of a biological sample based on the amount of cyanide in the sample by relating the amount of cyanide to the degree of conversion of the nitrile-group containing starting material.

59. The method of claim 2, wherein negative control samples and/or positive control samples are assayed with test samples to provide baselines for determining which biological samples have a desired activity.

60. The method of claim 1 or claim 2, wherein the method comprises assaying the catalytic activity of a material in a biological sample.

61. The method of claim 60, wherein the biological sample is derived from an environmental sample, a sample containing more than one organism, a sample comprising a mixed populations of organisms, an enriched sample, a sample from an isolated organism, a sample comprising a cultured organism or a sample comprising an uncultured organism.

62. The method of claim 60, wherein the biological sample comprises a microorganism existing in nature, a microorganism isolated from nature, a microorganism from a library, a clone from a library, an enzyme, a materials containing an enzyme, a cell, a DNA molecule, an RNA molecule or a living organism.

63. The method of claim 60, wherein the biological sample comprises a microorganism, a whole cell, an enzymes and/or a clone that comprises a sample from a mixed population library.

64. The method of claim 60, wherein the biological sample comprises a whole cell suspension or a clone from a mixed population library.

65. The method of claim 64, wherein the mixed population library is derived from a mixed population of organisms.

66. The method of claim 65, wherein the mixed population of organisms is derived from an environmental sample or an uncultivated population of organisms or a cultivated population of organisms.

67. The method of claim 1 or claim 2, wherein the method comprises use of a high throughput screening method.

68. The method of claim 67, wherein the high throughput screening method comprises a microarray or a fluorescence activated cell sorting (FACS).

69. The method of claim 68, wherein the microarray is GIGAMATRIX™.

70. The method of claim 54, wherein the catalyst which catalyzes the hydrolysis of nitrile groups in nitrile-group containing compounds is an enzymatic activity that catalyzes the hydrolysis of a compound selected from α-hydroxynitriles and aminonitriles.

71. The method of claim 54, wherein the catalyst which catalyzes the hydrolysis of nitrile groups in nitrile-group containing compounds is a nitrilase.

72. The method of claim 71, wherein the nitrilase comprises a nitrile hydratase, a hydroxynitrile lyase, or an oxynitrilase.

73. The method of claim 71, wherein the nitrilase comprises a sequence as set forth in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384 or 386.

74. The method of claim 71, wherein the nitrilase comprises a polypeptide encoded by a nucleic acid sequence as set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 374, 381, 383 or 385.

75. The method of claim 1, wherein the method is performed in a whole cell environment.

76. The method of claim 5, wherein the fluorescence detection technique comprises a fluorescence polarization, a time-resolved fluorescence, FRET, fluorescence activated cell sorting (FACS), HPLC or capillary electrophoresis (CE) technique.

77. A kit for determining if a biological sample has a particular activity comprising a substrate to be combined with the biological sample to form a reaction mixture, a derivatizing agent and an amine to be contacted with the reaction mixture to generate a fluorescent compound.

78. The kit of claim 77, wherein the derivatizing agent comprises a fluorescing agent.

79. The kit of claim 78, wherein the fluorescing agent comprises a naphthalene-2,3-dialdehyde (NDA) or equivalent or anthracene dicarboxyaldehyde (ADA) or equivalent.

80. The kit of claim 78, wherein the fluorescing agent comprises an o-phthalaldehyde (OPA) or equivalent.

81. The kit of claim 80, wherein the o-phthalaldehyde (OPA) is substituted at one or both of the 4 and 5 positions with substituents capable of enhancing the stability and fluorescence quantum of an isoindole product.

82. The kit of claim 81, wherein the substituents comprise a methoxy substituent, a dimethylamino substituent or both.

83. The kit of claim 78, wherein the fluorescing agent comprises a 3-(4-carboxybenzoyl)quinoline-2-carboxaldehyde (CBQCA, ATTO-TAG™).

84. The kit of claim 78, wherein the fluorescing agent comprises a composition having a formula comprising 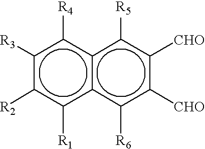
or variations thereof wherein one or more aromatic carbons are substituted with a heteroatom or a hetero group and/or one or both of the —CHO groups can be a —COR group, wherein R is selected from the group consisting of an alkyl group, an aryl group or an alkoxy group,
wherein the substituents R₁, R₂, R₃, R₄, R₅, R₆ are selected from one of the following:
(A) R₁ is selected from —H, an alkyl group, an aryl group, —N(CH₃)₂, —SO₃H, —NO₂, —SO₃⁻Na³⁰ and 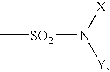
wherein X and Y may be the same or different and are independently selected from —H, an alkyl group, an aryl group and C1—C₈ alkyl groups, and R₂-R₆ are —H;
(B) R₁, R₄, R₅ and R₆ are —H, an alkyl group or an aryl group, and R₂ and R₃ are combined to form one of: 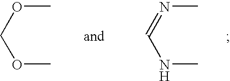
(C) R₁ and R₄ are independently selected from an alkyl group, an aryl group, —N(CH₃)₂ and 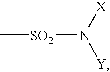
and R₂, R₃, R₅ and R₆ are —H, an alkyl group or an aryl group;
(D) R₁, R₂, R₃, and R₄ are —H, an alkyl group, an aryl group, and R₅ and R₆ are independently selected from an alkyl group, an aryl group, —OCH₃, 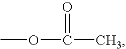
—OSi(CH₃)₂C₄H₉, and N(CH₃)₂; or
(E) R₁, R₄, R₅, and R₆ are —H, and R₂ and R₃ are independently selected from an alkyl group, an aryl group, —CH₃O , 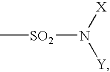
—SO₃H, —CO₂H, and salts thereof, and
(F) R₁, R₃, R₄, R₅ and R₆ are H an alkyl group or an aryl group, and R₂ is —(CH₃)₂N.

85. The kit of claim 84, wherein the heteroatom or hetero group comprises a nitrogen, an oxygen, a sulfur, a mercapto group, a thia group, a thio group, an aza group or an oxo group.

86. The kit of claim 78, wherein the fluorescing agent comprises a structure as set forth in FIG. 17, or, equivalent structures.
